(19) United States
(12) Patent Application Publication
(10) Pub. No.: US 2015/0211041 A1
(43) Pub. Date: Jul. 30, 2015

(54) PROGRAMMABLE NOR-BASED DEVICE FOR TRANSCRIPTION PROFILE ANALYSES

(71) Applicant: YEDA RESEARCH AND DEVELOPMENT CO., LTD., Rehovot (IL)

(72) Inventors: Ehud Y. Shapiro, Nataf (IL); Tom Ran, Rehovot (IL); Yehonatan Douek, Rehovot (IL)

(21) Appl. No.: 14/421,834

(22) PCT Filed: Aug. 14, 2013
(86) PCT No.: PCT/IL2013/050692

§ 371 Date: Feb. 15, 2015

Related U.S. Application Data
(60) Provisional application No. 61/683,816, filed on Aug. 16, 2012.

(51) Int. Cl. C12Q 1/02; C12N 15/63

(52) U.S. Cl. CPC C12Q 1/02; C12N 15/63

(57) ABSTRACT

An autonomous synthetic programmable device adapted to determine a cell state according to one or more different predefined markers, comprising a transcription machinery and a regulatory element for regulating said transcription machinery, said regulatory element comprising at least three different binding regions, each binding region binding a different transcription factor and each binding region corresponding to a different predefined marker, wherein the regulatory element permits said transcription machinery to provide a transcription output if and only if all three different transcription factors are absent and wherein each transcription factor is capable of blocking transcription by said transcription machinery and hence blocking said transcription output.

### FIELD OF THE INVENTION

The present invention, in at least some embodiments, provides a device, system and method for diagnosing a cell state, such as for example in relation to a disease, through integration of a plurality of molecular inputs without requiring pairwise interactions between these inputs.

### BACKGROUND OF THE INVENTION

Completion of the human genome sequence and technological advancements have made it possible to identify abnormal expression profiles in various diseases, including cancer¹⁻³. Transcription Factors (TFs) are proteins that regulate the expression of genes by binding to specific DNA sequences. In various diseases, coordinated de-regulation of expression can be found underlying the development or maintenance of the diseased states. For example, cancer cells alter their expression profile to promote uncontrolled proliferation and suppress cell death mechanisms⁴. Expression-based targeting, in which a therapeutic gene is expressed under the control of an impaired transcription factor, expressed solely in the target cells, holds the promise for smart drugs capable of differentiating diseased cells from healthy ones, and affecting the latter accordingly⁵. Treatments based on single disease markers have been demonstrated by delivering a therapeutic gene under the control of a promoter that can be activated by transcription factors that are overexpressed and/or constitutively activated in cancer cells in numerous tumor types⁶⁻¹⁰.

However, diagnosis based on a single input may be error prone. Integration of multiple disease indicators, such as transcription factors, is advantageous over a single indicator since it increases diagnosis accuracy and decreases the probability of falsely classifying healthy versus diseased cells. For these reasons, systems integrating multiple inputs have been implemented¹¹⁻¹⁶. These implementations are based on a constructive approach, in which the diagnostic computation is held in multiple steps. In the first step, each one of the disease markers controls a sub-component, such as a protein. In consecutive steps, sub-components repeatedly interact with each other to generate the final output, e.g., a reporter or a toxic protein, exclusively expressed in target diseased cells. Expanding these systems to a larger number of disease indicators requires addition of a large number of sub-components which iteratively hold the sub-computations. Thus, to increase the diagnostic accuracy of these systems, multiple complex biochemical reactions are required, and therefore scaling them up is expected to be difficult, thereby significantly reducing their clinical utility.

### SUMMARY OF THE INVENTION

Art known diagnostic systems based upon protein expression and other such disease indicators have many drawbacks, particularly regarding their scalability, because such diagnostic systems involve an “additive” approach, in which subcomponents are added to the diagnostic system until a differential diagnosis is achieved.

Surprising, the inventors of the present invention have overcome these drawbacks of the background art by using an “obstructing” approach, in which multiple disease indicators are integrated without requiring pairwise interactions. Instead, the present invention provides, in at least some embodiments, a device, referred to herein as a NOR-gate based device, which integrates one or more disease indicators, and which permits expression of an output if and only if all inputs are absent, with the proviso that pairwise interactions are permitted but not required. The device optionally and preferably harnesses only native cellular mechanisms to conduct computations.

In accordance with NOR gate's logic, the device preferably comprises a single regulatory element that can serve as an integrator of several inputs and enables the expression of an output if and only if all inputs are absent. According to at least some embodiments, the regulatory element comprises one or more potential binding regions, each corresponding to a specific pre-defined input. Preferably, each such binding input is sufficient for inhibiting the expression of the output by physically blocking the transcription machinery. The binding regions are preferably programmable and can utilize sequences of either prokaryotic TFs (transcription factors, such as lacI, which represses the expression of unnecessary proteins involved in the metabolism of lactose when the sugar is not available) or eukaryotic TFs (such as p53, which binds the promoter of Survivin, an apoptosis inhibitor highly expressed in most human tumors, and therefore represses its expression).

According to at least some embodiments, there is provided an autonomous synthetic programmable device that can diagnose a cell's state according to one or more predefined markers, comprising a transcription machinery and a regulatory element for regulating said transcription machinery, said regulatory element comprising one or more different binding regions, each binding region binding a different transcription factor and each binding region corresponding to a different predefined marker, wherein the regulatory element permits said transcription machinery to provide a transcription output if and only if all different predefined marker are absent and wherein each transcription factor is capable of blocking transcription by said transcription machinery and hence blocking said transcription output.

According to at least some embodiments, there is provided a plasmid, said plasmid comprising a genetic sequence adapted to be read by an RNA polymerase to cause expression of an output and one or more transcription factor binding sequence, wherein each transcription factor binding sequence binds a different transcription factor, i.e. predefined marker, and wherein said transcription factor binding sequences are selected such that said RNA polymerase can cause expression of said output if and only if none of said transcription factors bind to its respective transcription factor binding sequence, the plasmid being adapted to determine a molecular state of a cell when said cell is transfected with said plasmid.

According to at least some embodiments, there is provided a plasmid, said plasmid comprising a genetic sequence adapted to be read by an RNA polymerase to cause expression of an output and a transcription factor binding sequence, wherein said transcription factor binding sequence binds a transcription factor, i.e. predefined marker, and wherein said transcription factor binding sequence is selected such that said RNA polymerase can cause expression of said output if and only if said transcription factor does not bind to its respective transcription factor binding sequence, the plasmid being adapted to determine a molecular state of a cell when said cell is transfected with said plasmid.

Optionally, the plasmid comprises a plurality of transcription factor binding sequences, wherein each transcription factor binding sequence binds to a different transcription factor.

Optionally, the plurality of transcription factor binding sequences is at least three such binding sequences.

According to at least some embodiments, there is provided a plasmid, said plasmid comprising a genetic sequence adapted to be read by an RNA polymerase to cause expression of an output, a promoter sequence for controlling expression of said genetic sequence, a genetic sequence of a repressor protein and a plurality of transcription factor binding sequences, wherein each transcription factor binding sequence binds a different transcription factor, and wherein said transcription factor binding sequences are selected such that if said transcription factors, i.e. predefined marker bind to said transcription factor binding sequences, a repressor protein for binding to said promoter sequence and for blocking reading of said genetic sequence is expressed, such that said RNA polymerase can cause expression of said output if and only if at least one of said transcription factors does not bind to its respective transcription factor binding sequence, the plasmid being adapted to determine a molecular state of a cell when said cell is transfected with said plasmid.

According to at least some embodiments, there is provided a synthetic molecular device, comprising the above described plasmid and an RNA polymerase for binding to said plasmid and for causing said expression of said output, wherein said RNA polymerase is able to bind to the above described plasmid if and only if none of said transcription factors binds to said plasmid.

According to at least some embodiments, there is provided a plasmid, said plasmid comprising a genetic sequence adapted to be read by an RNA polymerase to cause expression of an output, a promoter sequence for controlling expression of said genetic sequence, a genetic sequence of a repressor protein and a plurality of transcription factor binding sequences, wherein each transcription factor binding sequence binds a different transcription factor, and wherein said transcription factor binding sequences are selected such that if said transcription factors do not bind to said transcription factor binding sequences, a repressor protein for binding to said promoter sequence and for blocking reading of said genetic sequence is expressed, such that said RNA polymerase can cause expression of said output if and only if each of said transcription factors binds to its respective transcription factor binding sequence, the plasmid being adapted to determine a molecular state of a cell when said cell is transfected with said plasmid.

According to at least some embodiments, there is provided a synthetic molecular device, comprising the above described plasmid and an RNA polymerase for binding to said plasmid and for causing said expression of said output, wherein said RNA polymerase is able to bind to the above described plasmid if and only if all of said transcription factors bind to said plasmid.

According to at least some embodiments, there is provided a cell having a molecular state, comprising the above described plasmid, wherein the molecular state of the cell is determined according to whether said RNA polymerase causes said output to be produced.

According to at least some embodiments there is provided a method for diagnosing a molecular state of a cell, comprising transfecting the cell with the plasmid as described herein; and detecting a presence or absence of said output, wherein said presence or absence of said output determines said molecular state of said cell.

Optionally, if said output is produced, the molecular state of the cell is a disease state, preferably comprising cancer. Alternatively and optionally, if said output is produced, the molecular state of the cell is a disease state, preferably comprising cancer.

Optionally and preferably, the output comprises one or more of a protein or an RNA molecule. Optionally, said RNA molecule comprises a miRNA (micro RNA) molecule.

Optionally and preferably, said transcription factor binding region(s) comprise regions that are located in one or more of downstream, upstream or in-between conserved regions, or a combination thereof.

Also optionally and preferably, the transcription factor(s) comprise only innate transcription factor(s) already present in a cell, such that optionally the plasmid does not enable expression of any transcription factors or alternatively only enables expression of a single output protein or RNA molecule.

Also optionally and preferably, the transcription factor(s) comprise prokaryotic transcription factors or eukaryotic transcription factors. The prokaryotic transcription factor(s) optionally comprise any repressing transcription factor such as TetR, LacI, or λ-Repressor. The eukaryotic transcription factor(s) optionally comprise any repressing or activating transcription factor such as p53, E2F, or FOXO.

Motivated to increase diagnosis precision, devices that integrate multiple disease markers have been implemented based on various molecular tools. As simplicity is key to future in-vivo applications, as described herein in various embodiments, a molecular device was designed that a) integrates multiple inputs without requiring pairwise interactions, and b) harnesses only mechanisms that cells natively use. The inventive synthetic NOR-based programmable device, operating via a biochemical obstructing approach rather than on a constructive approach, is (for example and without limitation) capable of differentiating between prokaryotic cell strains based on their unique expression profile. The device's programmability allows context-dependent selection of the inputs being sensed, and of the expressed output, thus, holding great promise in future biomedical applications.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the NOR-gate and its molecular implementation;

FIG. 2 shows molecular implementation of basic logic gates assembled by the NOR gate;

FIG. 3 shows various control results, indicating that the experimental system behaved as expected under various conditions;

FIG. 4 shows the AND-gate's control experiments and system kinetics;

FIG. 5 shows scalability of a NOR-gate VS. AND-gate based systems;

FIG. 6 shows expression based selective induction of apoptosis;

FIG. 7 shows a table of the nomenclature used; and

FIG. 8 shows the derivative plasmids and their nomenclature.

### DESCRIPTION OF SOME EMBODIMENTS

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Methods

Strains.

All studies were performed using four different DH5α E. coli strains, genomically expressing the four inputs combinations, none, LacI, TetR and Lad and TetR, termed DH5α, DH5αZi, DH5αZr and DH5αZl, respectively. DH5αZr (chromosomal TetR integration) was achieved as follows: DH5αZr was prepared via chromosomal integration procedure as follows: The TetR gene was integrated in a DH5α E. coli strain that carries in its chromosome the attB site via Int mediated site specific recombination. For this, plasmid pZS4Int-tetR together with pIntAssist were used. pIntAssist carries a temperature sensitive origin of replication and upon heat treatment was lost after the integration procedure, i.e. the resulting strain carries a Spectinomycin resistance cassette in the chromosome only. A respective protocol can be found by the supplier of the pZ system (more details can be found on the website http://expressys.com/).

Media.

Lysogeny broth (LB) plates with appropriate antibiotics were obtained from the bacteriology services (Weizmann Institute) and prepared as described (Sambrook J, R. D. Molecular Cloning: A laboratory manual, 3rd ed. Cold Spring Harbor Laboratory Press, New York (2001)). Strains were grown in Lysogeny broth (LB) medium supplied by the Weizmann institute bacteriology unit and were grown overnight at 37° C. with 250 rpm shaking. The cultures were diluted 1:100 into 200 n1 of medium in a 96-well plate with different combinations of antibiotics and/or inducers; 34 μg/ml 1:1000 50 mg/ml chloramphenicol and/or 50 μg/ml kanamycin and/or 100 μg/ml Ampicillin and/or 50 μg/ml Spectomycin and/or IPTG 1 mM and/or anhydrotetracycline 100 ng/ml.

Plasmids.

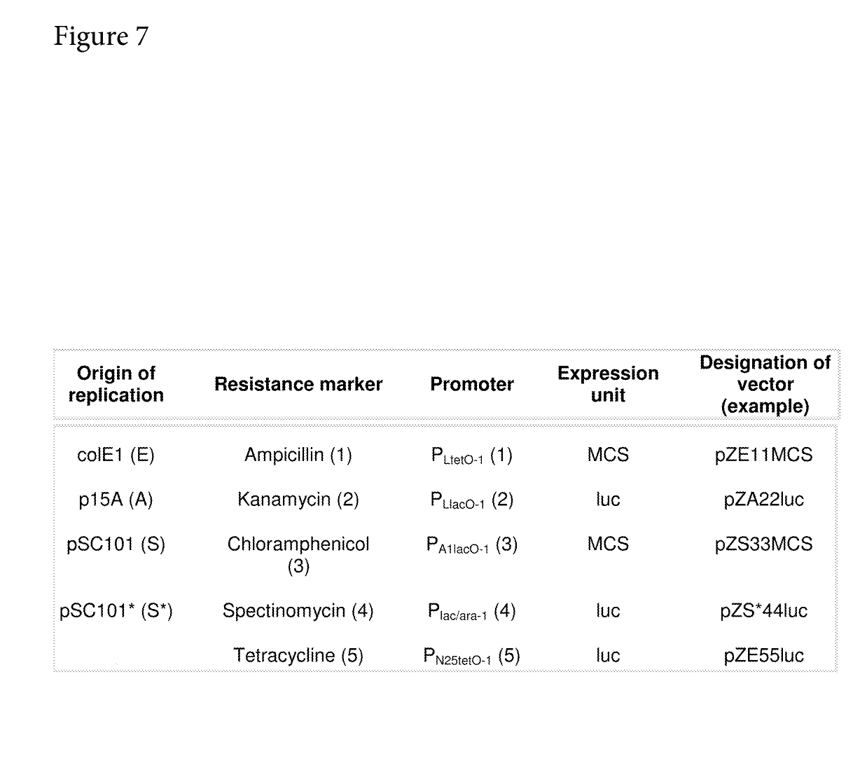

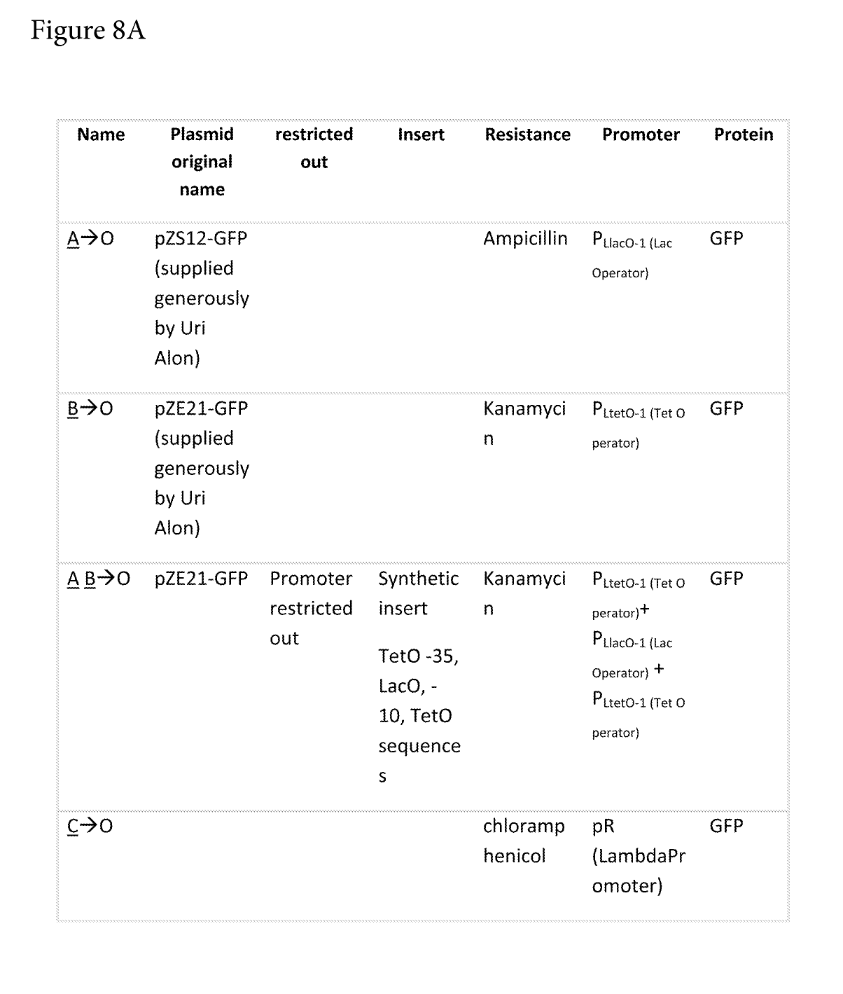

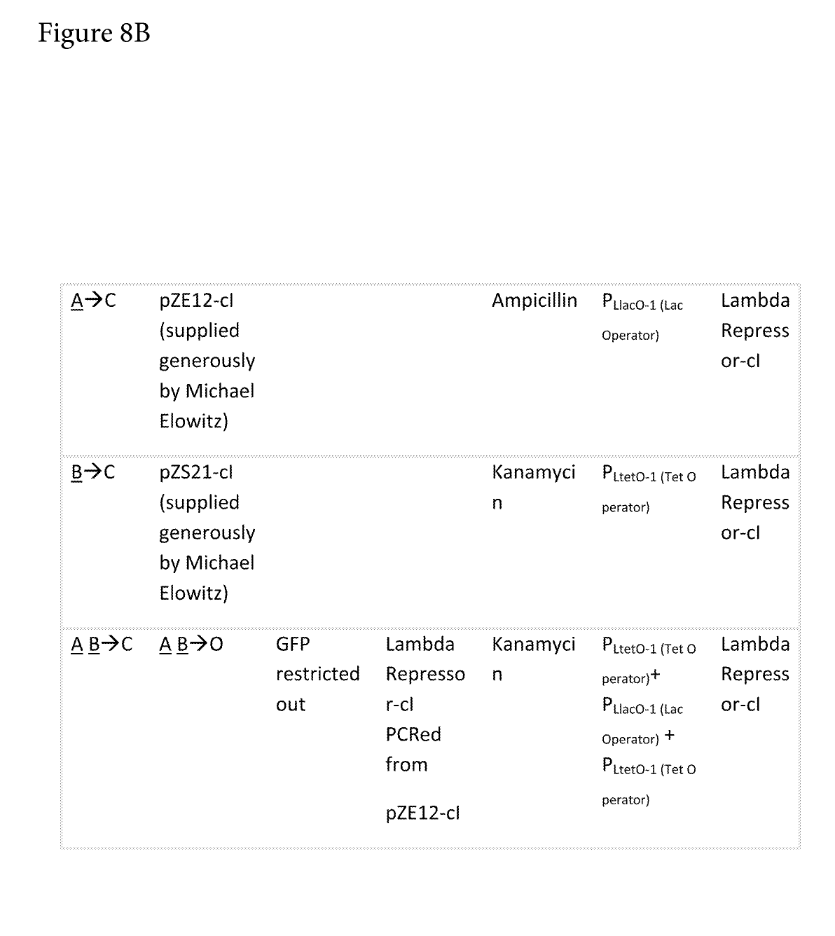

All plasmids are based on the components of the pZ Expression System and its nomenclature is as follows: The letter (E, A, S, S*) denotes the origin of replication. The first number indicates the resistance marker (1 to 5). The second number (1 to 5) defines the promoter controlling the transcription of the gene of interest. The MCS or the description of the gene of interest, e.g. GFP, follows this code as exemplified. The nomenclature can be found in FIG. 7, and the derivative plasmids and their nomenclature used herein can be found in FIG. 8. The inventors thank the kind members of the laboratories of Uri Alon and Michael Elowitz for sharing their wisdom and plasmids.

Liquid Handling and Measurements.

Assembly, execution and readout of the experiments, i.e., liquid handling, orbital shaking, growth in stable 37° C. temperature, were done on a Tecan Freedom® 2000 robot controlled by in-house developed software. Fluorescence signals were read by a Tecan Infinite® 200 microplate-reader: GFP (Excitation Wavelength: 497 nm Emission Wavelength: 535 nm). mCherry (Excitation Wavelength: 587 nm Emission Wavelength: 614 nm). Reaction's components: *) LB. *) Bacteria strain, expressing one of the four desired input combinations (none, LacI, TetR and Lad and TetR), and transformed with one or more of the plasmids implementing desired gates. *) Appropriate antibiotics according to FIGS. 7 and 8.

### Results

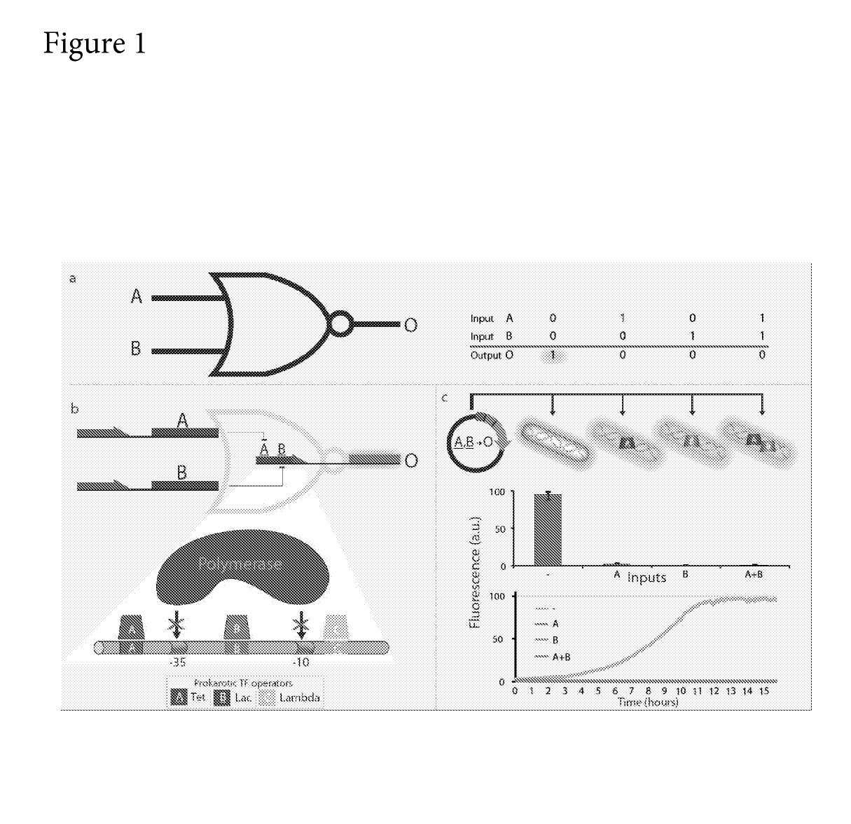

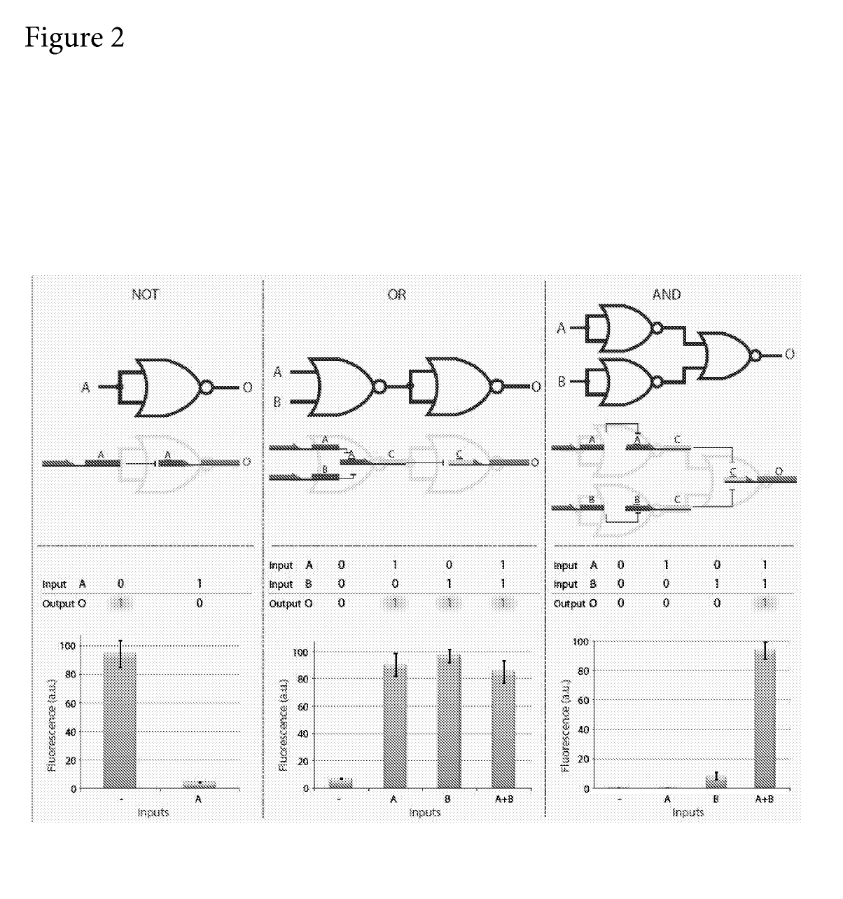

FIG. 1 shows the NOR-gate and its molecular implementation (FIG. 1) and the molecular implementation of basic logic gates assembled by the NOR gate (FIG. 2).

Turning now to FIG. 1, which shows the NOR-gate and its molecular implementation, in accordance with NOR gate's logic, as can be seen in FIG. 1a, a single regulatory element was designed that can serve as an integrator of several inputs and enables the expression of an output if and only if all inputs are absent (FIG. 1b). The regulatory element is comprised of several potential binding regions, each corresponding to a specific pre-defined input (FIG. 1b, balloon). One binding input is sufficient for inhibiting the expression of the output by physically blocking the transcription machinery. The binding regions are programmable and can utilize sequences of either prokaryotic TFs (such as lad, which represses the expression of unnecessary proteins involved in the metabolism of lactose when the sugar is not available) or eukaryotic TFs (such as p53, which binds the promoter of Survivin, an apoptosis inhibitor highly expressed in most human tumors, and therefore represses its expression).

FIG. 1a shows the universal NOR-gate and its truth table. FIG. 1b shows the molecular implementation of the NOR synthetic genetic circuit. A single regulatory element can be repressed by either one of several potential inputs. If and only if none are present, the RNA polymerase can attach to its binding site resulting in the GFP output's expression (A and B and A and B represent the TFs Lad and TetR and their corresponding potential binding regions Lac-Operator and Tet-Operator, respectively. O represents GFP). The integrator is comprised of arbitrary regions, located downstream, upstream and in-between conserved regions, responsible for recruiting the transcription machinery (e.g., the RNA polymerase and its -35-10 recruiting sequences). The arbitrary regions can be assigned with binding regions for TFs. This design applies for prokaryotic TFs (e.g., TetR, LacI, 2-Repressor, etc.) as well as for Eukaryotic TFs by principal (e.g., p53, E2F, FOXO, etc.).

FIG. 1C shows the truth table of the four E. coli strains used to test the NOR synthetic genetic circuit, each genomically expressing one of the four possible input combinations. The NOR-gate plasmid was transformed into the four different strains. As can be seen, only the strain presenting none of the inputs resulted in a ‘1’ signal while the rest, presenting one or two inputs, resulted in a ‘0’ signal, in accordance to the NOR's truth table. Kinetic results are also shown, exhibiting efficient digital behavior over time—high signal strength while maintaining no signal leakage. Arbitrary unit (a.u.) is calculated as fluorescence/O.D₂. Fluorescence values and their error bars are calculated as mean±s.d. from three experiments.

FIG. 2 shows molecular implementation of basic logic gates assembled by the NOR gate. A, B and C and A, B and C represent the TFs LacI, TetR and 2-Repressor and their corresponding potential binding regions Lac-Operator, Tet-Operator and λ-Operator, respectively. O represents GFP.

FIG. 2a describes an exemplary NOT gate. If and only if (IFF) A is present, its corresponding promoter which controls the expression of the output protein is blocked, resulting in a ‘0’ output signal.

FIG. 2b describes an exemplary OR gate. IFF both A and B are absent, the expression of C is enabled, which in turn represses its promoter that controls the expression of the output protein, resulting in a ‘0’ output signal.

FIG. 2c describes an exemplary AND gate. Both A and B are needed to repress C, which in turn controls the expression of the output protein. Thus, IFF both input signals are ‘1’ the output signal is ‘1’. Arbitrary unit (a.u.) is calculated as fluorescence\O.D₂. Fluorescence values and their error bars are calculated as mean±s.d. from three experiments.

The NOR Gate.

This design was demonstrated in prokaryotic cells. The illustrative, exemplary integrator, according to at least some embodiments of the present invention, is capable of differentiating between four strains of E. coli, genomically expressing different logic combinations of two common TFs: NOR(A=0, B=0), XOR(A=1, B=0 or A=0, B=1) and AND(A=1, B=1). To test this ability the NOR-gate plasmid was transformed into the four different strains, as depicted in FIG. 1c. Only in strains expressing at least one of the TFs, the RNA polymerase is blocked from attaching to its binding site and the output protein is not expressed. All inputs and outputs are of the same type, i.e., TFs, allowing composition of logical circuits. The integrator controls the expression of another TF, which can serve as an input to another logic gate. To further test the NOR-gate in terms of robustness, efficiency and digital behavior, three basic logic gates NOT, OR and AND were implemented as shown in FIG. 2 and as described below.

NOT Gate.

The NOT gate is based upon a rather straight-forward signal inverter. If and only if input A's signal is ‘1’, i.e. repressor TF that represents input A is present, its corresponding promoter which controls the expression of the output protein is blocked, resulting in a ‘0’ output signal. As seen in FIG. 2a, the output protein was expressed only in strains lacking input A, corresponding to a NOT-gate's logic.

OR Gate.

The OR gate plasmid was derived from the previously constructed NOR gate, in which the output protein was replaced with an intermediate repressor, C. The resulting plasmid is comprised of a promoter incorporating the binding regions of inputs A and B, and controls the expression of C in a NOR fashion. Based on the abstract digital logical representation, in which the OR gate is formed by inverting the NOR gate's signal, an additional element was added, in which the output protein is controlled by the inverting repressor, C. If and only if both A and B are absent, repressor C is expressed and the output protein is blocked from expression. As seen in FIG. 2b, the output protein was expressed in strains containing either input A, input B, or both—corresponding to an OR-gate's logic.

AND Gate.

In order to implement the AND gate, the intermediate repressor C was placed under the control of both inputs, A and B, in an independent manner. The output protein was placed under the control of the C repressor. If and only if repressor C is absent, the output protein is expressed. Repressor C's absence is dependent on both input A and input B's presence. Overall, as seen in FIG. 2c, the output protein was expressed only in strains containing both input A and input B—corresponding to an AND gate's logic.

As can be seen all gates maintained robust and digital behavior, exhibiting very low signal leakage and keeping a high signal yield and strength.

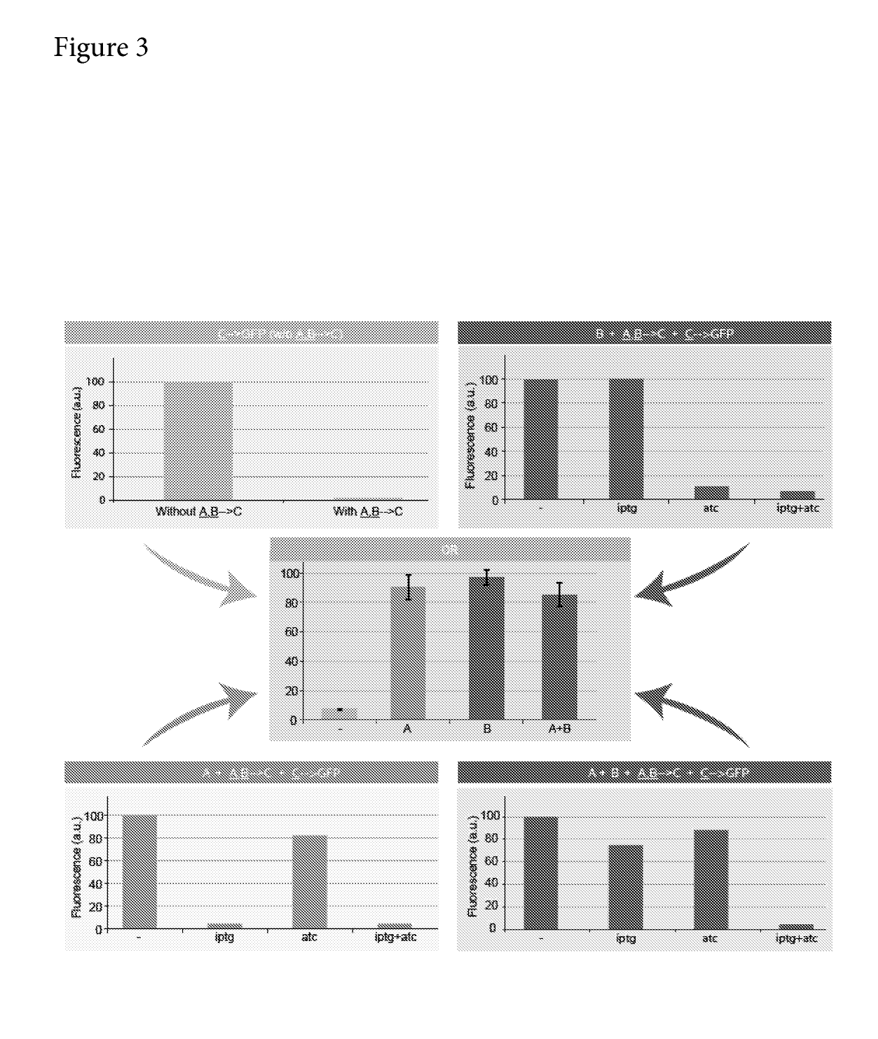

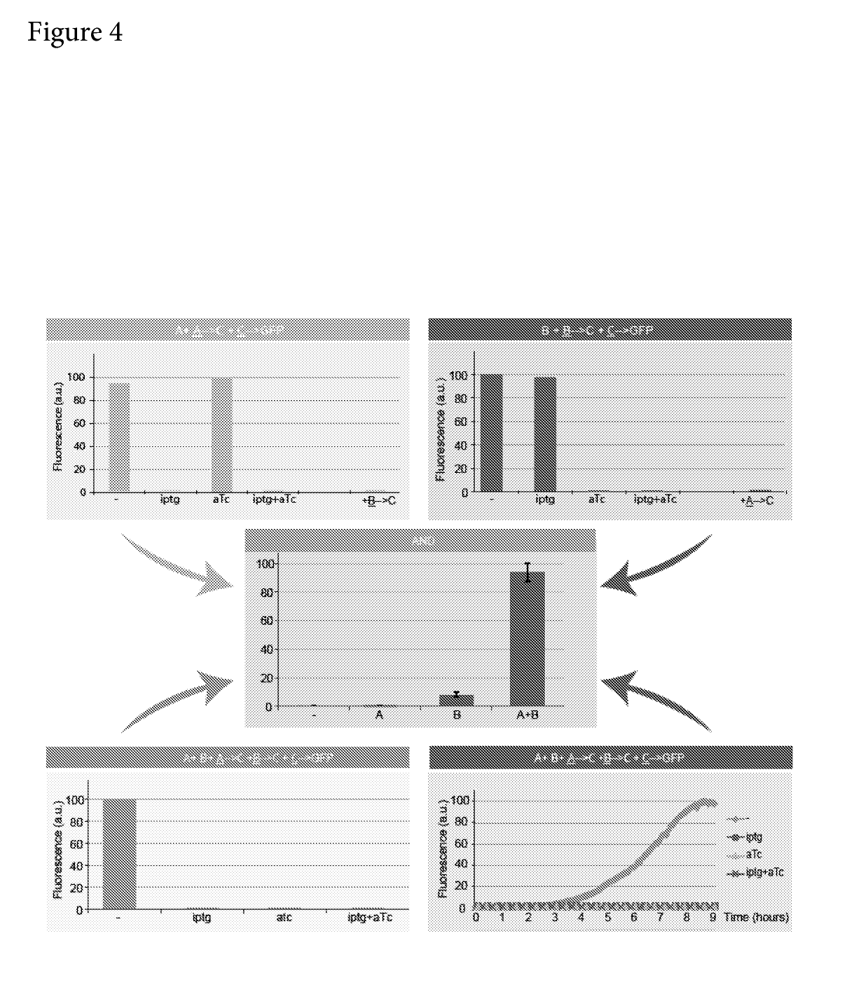

Control experiments, including kinetics of the system, were performed according to the previously described methods. The results are shown in FIGS. 3 and 4.

FIG. 3 shows various control results, indicating that the experimental system behaved as expected under various conditions.

Upper left panel, λ-Operator & λ-Repressor. Bacterial strain which normally expresses GFP under the control of a promoter harboring the λ-Operator (potentially repressible by the λ-Repressor). Left and Right bar—The depicted strain was either transformed or not with a plasmid carrying a promoter incorporating the Lac Operator and the Tet Operator controlling the expression of the λ-Repressor. The promoter is repressible by either Lad or TetR, which are not expressed by the host strain. As can be seen by the Right bar, the fluorescence was quenched only when the plasmid expressing the λ-Repressor was present, attesting that the plasmid indeed expresses a functional λ-Repressor.

Lower left panel, Lad expressing bacteria. To counteract Lad and check for potential unwanted cross-talks, four different conditions were tested—by adding to the medium nothing, IPTG, aTc or IPTG and aTc (IPTG should counteract Lad solely, while aTc should counteract TetR solely). As can be seen, only when Lad was active, i.e., no IPTG was present in the medium, Lad repressed the expression of the λ-Repressor which in turn did not repress its downstream promoter which in turn expressed GFP (first and third bars).

Upper right panel, TetR expressing bacteria. To counteract TetR and check for potential unwanted cross-talks, four different conditions were tested—by adding to the medium nothing, IPTG, aTc or IPTG and aTc (IPTG should counteract Lad solely, while aTc should counteract TetR solely). As can be seen, only when TetR was active, i.e., no aTc was present in the medium, TetR repressed the expression of the λ-Repressor which in turn did not repress its downstream promoter which in turn expressed GFP (first and second bars).

Lower right panel, Lad & TetR expressing bacteria. To counteract Lad and/or TetR, four different conditions were tested—by adding to the medium nothing, IPTG, aTc or IPTG and aTc (IPTG should counteract Lad solely, while aTc should counteract TetR solely). As can be seen, only when Lad and TetR were counteracted by the addition of both IPTG and aTc, neither transcription factor repressed the expression of the λ-Repressor which in turn repressed its downstream promoter which in turn expressed GFP (fourth bar).

FIG. 4 shows the AND-gate's control experiments and system kinetics.

Upper left panel, Lad expressing bacteria. To counteract Lad and check for potential unwanted cross-talks, four different conditions were tested—by adding to the medium nothing, IPTG, aTc or IPTG and aTc (IPTG should counteract Lad solely, while aTc should counteract TetR solely). As can be seen, only when Lad was active, i.e., no IPTG was present in the medium, Lad repressed the expression of the λ-Repressor which in turn did not repress its downstream promoter which in turn expressed GFP (first and third bars). The fifth condition tested the addition of the plasmid carrying a promoter incorporating the Tet Operator controlling the expression of the λ-Repressor. Addition of the plasmid should result the GFP's quenching—given that the strain does not express TetR, λ-Repressor's expression is enabled. λ-Repressor will in turn repress the expression of the GFP.

Upper right panel, TetR expressing bacteria. To counteract TetR and check for potential unwanted cross-talks, four different condition were tested—by adding to the medium nothing, IPTG, aTc or IPTG and aTc (IPTG should counteract Lad solely, while aTc should counteract TetR solely). As can be seen, only when TetR was active, i.e., no aTc was present in the medium, TetR repressed the expression of the λ-Repressor which in turn did not repress its downstream promoter which in turn expressed GFP (first and second bars). The fifth condition tested the addition of the plasmid carrying a promoter incorporating the Lac Operator controlling the expression of the λ-Repressor. Addition of the plasmid should result the GFP's quenching—given that the strain does not express LacI, λ-Repressor's expression is enabled. λ-Repressor will in turn repress the expression of the GFP.

Lower left & right panels, Lad & TetR expressing bacteria. Left panel. To counteract Lad and/or TetR, four different conditions were tested—by adding to the medium nothing, IPTG, aTc or IPTG and aTc (IPTG should counteract Lad solely, while aTc should counteract TetR solely). As can be seen, when either Lad or TetR, or both were counteracted by either the addition of IPTG and/or aTc, λ-Repressor was expressed which in turn repressed its downstream promoter controlling the expression of the GFP (first, second and third bars). Right panel. Kinetic results.

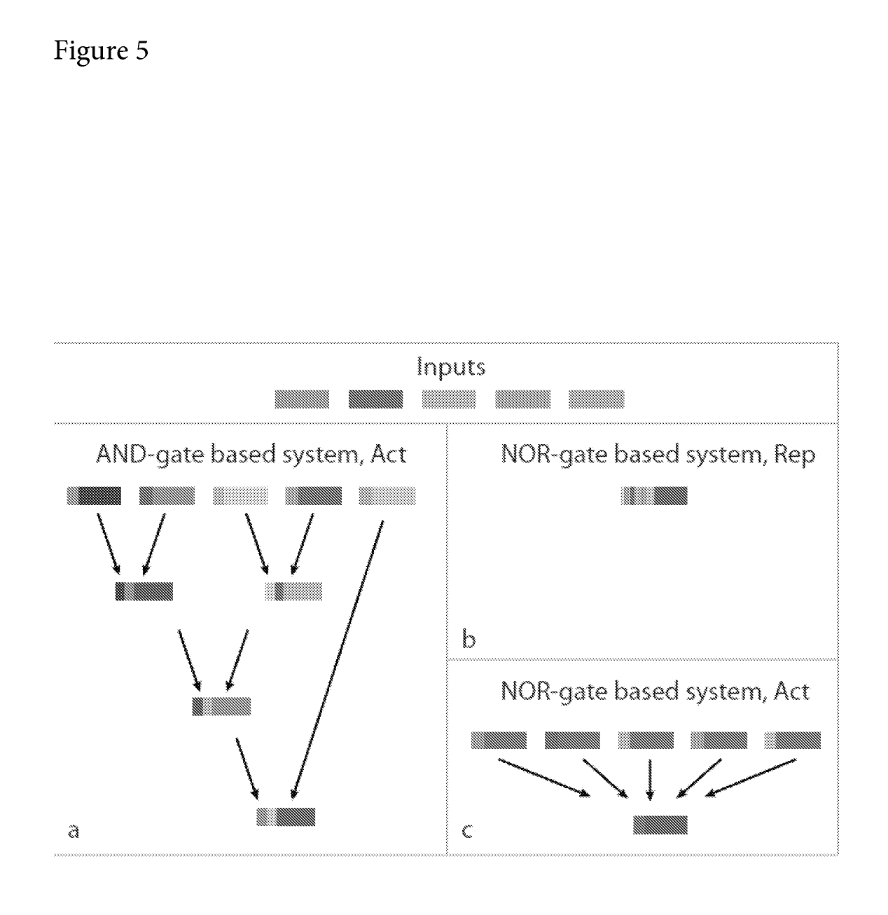

FIG. 5 shows scalability of a NOR-gate VS. AND-gate based systems. Corresponding colors represent a TF with its corresponding promoter a) AND-gate based systems, such as systems based on the two-hybrid system (which use activator TFs), require co-interaction of pairs of inputs through iterative sub-computations. b) In a NOR-gate based system, following a design such as the one we have suggested, inputs directly control the output. The addition of new inputs does not require new intermediate proteins or interactions, enabling simple scalability. c) AND-gate based systems accept as input the activating TFs controlling the expression of the analyzed proteins. Panel c, depicts a NOR-based design that by utilizing a signal inverter, is equivalent to the design depicted in panel a. in terms of the input it accepts and the corresponding output. In both designs, if and only if all activating TFs are present, the final output will be expressed, only that the NOR-gate design spares the co-interactions required by the AND-gate's design.

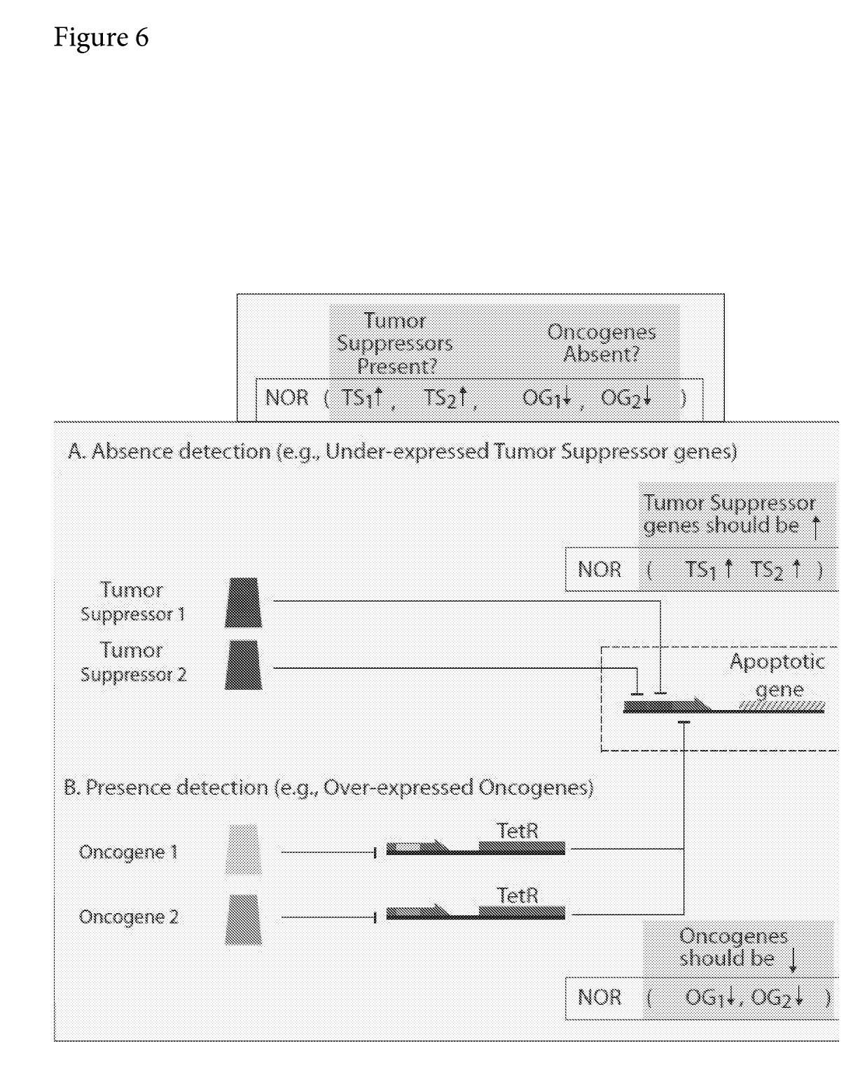

FIG. 6 shows expression based selective induction of apoptosis. High levels of tumor suppressors and low levels of oncogenes are characteristic of healthy normal cells. In the NOR-based circuit presented, it suffices for one tumor suppressor or oncogenes to be normally expressed to suppress the suicide gene. In other words, for the apoptosis inducing gene to be expressed, all tumor suppressors should be suppressed and all oncogenes over-expressed.

### Discussion

The above experiments show implementation of a dual-repressed promoter, serving as a NOR gate, along with a complete set of Boolean gates (NOT, OR & AND) in prokaryotic cells. The system is modular and programmable by design—any repressing TF can be used as its input, and any gene of interest can be set as the expressed output. The devices, cells and plasmids as described herein permit direct and easy composition of basic logic gates into cascadable circuits, unlike systems based on tRNA¹⁸, aptamers or RNA alternative splicing¹¹, and microRNAs and RNA interference^{15,19,20}. A system possessing these features—input and output modularity, programmability and cascadability—allows accurate targeting of desired cells without falsely targeting other cells.

The exemplary NOR-based design described herein can be scaled to multiple inputs while maintaining a simple molecular implementation by forsaking pairwise interaction of the different individual inputs. Unlike AND-gate based systems¹³, which require pairwise interactions of inputs through iterative sub-computations (as depicted in FIG. 5), the exemplary NOR-based design is based on the direct integration of different inputs, where each input directly and independently controls the output gene, in parallel with the other inputs. In addition, the system is based on an obstructive approach, e.g., repressing TFs that interfere with the regular regulatory machinery by steric blockage, rather than a constructive approach, e.g., protein—protein interactions which is not easy to scale.

Tasmir et al.¹⁷ recently demonstrated a genetic NOR gate based on the concatenation of two potentially repressible tandem promoters in E. coli. Either promoter, if in an unrepressed state, can solely suffice to drive the expression of a downstream repressor, which in turn can repress its corresponding downstream output gene. However, the present invention has many advantages over this system, described herein without wishing to be limited to a closed list. In terms of scalability, given that promoters are large entities, only a small number can be concatenated, since each added promoter will have to be farther from the transcriptional start site. This is particularly relevant for future medical applications given that mammalian cells' promoters are of much greater magnitude. In contrast, the repression operators (approximately 20 bases) are significantly smaller than promoters and therefore many can be concatenated within one promoter. Additionally, in the system of Tasmir et al.¹⁷, the inputs are two chemical external inducers incubated in the culture tubes together with the bacteria. These inducers can bind and inhibit the two TFs repressors that repress the two tandem promoters. If and only if the two external inducers are absent the output gene was expressed. External inputs accommodated Tasmir et al.¹⁷ goal of interconnecting individual E. coli colonies via chemical components functioning as the ‘wires’. However, the changes and anomalies underlining various diseases start and subside with endogenous intra-cellular changes⁴ (such as the deregulation of TFs levels).

Therefore, for the goal of cell-state diagnosis computing and for the present invention, the better choice is to use internal inputs. Delivery of the NOR circuit using traditional methods (such as transfection²⁵) into all cells (target and normal) will allow the circuit to sense and analyze these intra-cellular inputs present inside the cell. Accordingly, an integrator was designed that accepts innate TFs as inputs and computes NOR-based logic gates with them. Together, these features offer an advance over previous approaches as they simplify the biochemical reactions underlying the computation and increase the feasibility to operate in a biological environment.

The above described system's abilities were tested in prokaryotic cells which are far less complex than mammalian cells. However, various embodiments of the present invention may also optionally be applied to diagnosis of disease indicators in mammalian cells as it is based on: a native cellular machinery; a destructive approach; and, can analyze both over-expressed TFs (such as oncogenes), and under-expressed TFs (such as tumor suppressors). When detecting the absence of tumor suppressors, it suffices for one tumor suppressor (which normally should be present) to directly attach onto its corresponding potential binding region and inhibit the expression of a protein which induces apoptotic cell death, as shown in FIG. 6a.

When detecting the presence of oncogenic TFs, the over-expressed oncogenes converge to inhibit the expression of an intermediate repressor which in turn inhibits the expression of the output protein. One normally absent oncogene suffices to inhibit the expression of the output protein, as shown in FIG. 6b. Thus, in accordance with the NOR gate truth table, if and only if all inputs are aberrantly expressed, i.e., all tumor suppressors are absent and all oncogenes are present, the output is expressed. The system presented in this work demonstrates how the NOR gate can analyze TF inputs based on their digital presence or absence (as opposed to being able to analyze any analog or gradual level of expression). Although analog gradual de-regulation is more common than digital exclusive presence or absence, it is the last that holds the promise for cancer-specific gene therapies. Digital, i.e., unique and distinct markers, enable greater specificity and optimized target versus non-target cells discrimination. And indeed, cancer-specific gene therapies' based on this digital absence or presence principle, have already been clinically tested in numerous cancer types⁶⁻¹⁰. In these transcriptionally targeted gene therapies, a digital TF⁴ exclusively present in target cells, while absent in normal cells, solely controls the expression of a therapeutic gene. Thus, corresponding exclusive expression in target cells and not in normal cells is achieved.

Scaling up the number of sensed inputs, while sensing both aberrantly present (e.g., oncogenes) and aberrantly absent (e.g., tumor suppressors) TFs, vastly broadens the repertoire of potential markers that can be analyzed. A mammalian system based on this design may allow analyzing the presence or absence of numerous cancer-related TFs and the induction of cells death if all TFs were aberrantly expressed, and therefore may have important future biological and medical applications.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### REFERENCES

1 Lo, H. W., Day, C. P. & Hung, M. C. Cancer-specific gene therapy. Advances in genetics 54, 235-255 (2005).
2 Ramaswamy, S. et al. Multiclass cancer diagnosis using tumor gene expression signatures. Proceedings of the National Academy of Sciences of the United States of America 98, 15149-15154 (2001).
3 Chung, C. H., Bernard, P. S. & Perou, C. M. Molecular portraits and the family tree of cancer. Nature genetics 32 Suppl, 533-540 (2002).
4 Vogelstein, B. & Kinzler, K. W. Cancer genes and the pathways they control. Nature Medicine 10, 789-799 (2004).
5 DeFatta, R. J., Li, Y. & De Benedetti, A. Selective killing of cancer cells based on translational control of a suicide gene. Cancer gene therapy 9, 573-578 (2002).
6 Venkataraman, S., Dirks, R. M., Ueda, C. T. & Pierce, N. A. Selective cell death mediated by small conditional RNAs. Proceedings of the National Academy of Sciences of the United States of America 107, 16777-16782 (2010).
7 Pandha, H. S. et al. Genetic prodrug activation therapy for breast cancer: A phase I clinical trial of erbB-2-directed suicide gene expression. Journal of clinical oncology: official journal of the American Society of Clinical Oncology 17, 2180-2189 (1999).
8 Terazaki, Y. et al. An optimal therapeutic expression level is crucial for suicide gene therapy for hepatic metastatic cancer in mice. Hepatology 37, 155-163 (2003).
9 Song, J. S. & Kim, H. P. Adenovirus-mediated HSV-TK gene therapy using the human telomerase promoter induced apoptosis of small cell lung cancer cell line. Oncology reports 12, 443-447 (2004).
10 Song, J. S. et al. Adenovirus-mediated suicide gene therapy using the human telomerase catalytic subunit (hTERT) gene promoter induced apoptosis of ovarian cancer cell line. Bioscience, Biotechnology and Biochemistry 67, 2344-2350 (2003).
11 Culler, S. J., Hoff, K. G. & Smolke, C. D. Reprogramming cellular behavior with RNA controllers responsive to endogenous proteins. Science 330, 1251-1255 (2010).
12 Nissim, L., Beatus, T. & Bar-Ziv, R. An autonomous system for identifying and governing a cell's state in yeast. Physical biology 4, 154-163 (2007).
13 Nissim, L. & Bar-Ziv, R. H. A tunable dual-promoter integrator for targeting of cancer cells. Molecular systems biology 6, 444 (2010).
14 Rinaudo, K. et al. A universal RNAi-based logic evaluator that operates in mammalian cells. Nature Biotechnology 25, 795-801 (2007).
15 Leisner, M., Bleris, L., Lohmueller, J., Xie, Z. & Benenson, Y. Rationally designed logic integration of regulatory signals in mammalian cells. Nature nanotechnology 5, 666-670 (2010).
16 Xie, Z., Wroblewska, L., Prochazka, L., Weiss, R. & Benenson, Y. Multi-Input RNAi-Based Logic Circuit for Identification of Specific Cancer Cells. Science 333, 1307-1311 (2011).
17 Tamsir, A., Tabor, J. J. & Voigt, C. A. Robust multicellular computing using genetically encoded NOR gates and chemical ‘wires’. Nature 469, 212-215 (2011).
18 Anderson, J. C., Voigt, C. A. & Arkin, A. P. Environmental signal integration by a modular AND gate. Molecular systems biology 3, 133 (2007).
19 Rinaudo, K. et al. A universal RNAi-based logic evaluator that operates in mammalian cells. Nat Biotechnol 25, 795-801 (2007).
20 Deans, T. L., Cantor, C. R. & Collins, J. J. A tunable genetic switch based on RNAi and repressor proteins for regulating gene expression in mammalian cells. Cell 130, 363-372 (2007).

1. A plasmid, said plasmid comprising a genetic sequence adapted to be read by an RNA polymerase to cause expression of an output and one or more transcription factor binding sequences, wherein if more than one transcription factor binding sequence is present, each transcription factor binding sequence binds a different transcription factor, and wherein said transcription factor binding sequences are selected such that said RNA polymerase can cause expression of said output if and only if none of said transcription factors bind to its respective transcription factor binding sequence, the plasmid being adapted to determine a molecular state of a cell when said cell is transfected with said plasmid.

2. A plasmid, said plasmid comprising a genetic sequence adapted to be read by an RNA polymerase to cause expression of an output and a transcription factor binding sequence, wherein said transcription factor binding sequence binds a transcription factor, and wherein said transcription factor binding sequence is selected such that said RNA polymerase can cause expression of said output if and only if said transcription factor does not bind to its respective transcription factor binding sequence, the plasmid being adapted to determine a molecular state of a cell when said cell is transfected with said plasmid.

3. The plasmid of claim 1, comprising a plurality of transcription factor binding sequences, wherein each transcription factor binding sequence binds to a different transcription factor.

4. The plasmid of claim 3, wherein said plurality of transcription factor binding sequences is at least three such binding sequences.

5. A plasmid, said plasmid comprising a genetic sequence adapted to be read by an RNA polymerase to cause expression of an output, a promoter sequence for controlling expression of said genetic sequence, a genetic sequence of a repressor protein and a plurality of transcription factor binding sequences, wherein each transcription factor binding sequence binds a different transcription factor, and wherein said transcription factor binding sequences are selected such that if said transcription factors bind to said transcription factor binding sequences, a repressor protein for binding to said promoter sequence and for blocking reading of said genetic sequence is expressed, such that said RNA polymerase can cause expression of said output if and only if at least one of said transcription factors does not bind to its respective transcription factor binding sequence, the plasmid being adapted to determine a molecular state of a cell when said cell is transfected with said plasmid.

6. A molecular device, comprising said plasmid of claim 1 and an RNA polymerase for binding to said plasmid and for causing said expression of said output, wherein said RNA polymerase is able to bind to said plasmid if and only if none of said transcription factor(s) binds to said plasmid.

7. A plasmid, said plasmid comprising a genetic sequence adapted to be read by an RNA polymerase to cause expression of an output, a promoter sequence for controlling expression of said genetic sequence, a genetic sequence of a repressor protein and a plurality of transcription factor binding sequences, wherein each transcription factor binding sequence binds a different transcription factor, and wherein said transcription factor binding sequences are selected such that if said transcription factors do not bind to said transcription factor binding sequences, a repressor protein for binding to said promoter sequence and for blocking reading of said genetic sequence is expressed, such that said RNA polymerase can cause expression of said output if and only if each of said transcription factors binds to its respective transcription factor binding sequence, the plasmid being adapted to determine a molecular state of a cell when said cell is transfected with said plasmid.

8. A molecular device, comprising said plasmid of claim 7 and an RNA polymerase for binding to said plasmid and for causing said expression of said output, wherein said RNA polymerase is able to bind to said plasmid if and only if all of said transcription factors bind to said plasmid.

9. A cell having a molecular state, comprising the plasmid of claim 1, wherein the molecular state of the cell is determined according to whether said RNA polymerase causes said output to be produced.

10. A method for diagnosing a molecular state of a cell, comprising transfecting the cell with the plasmid of claim 1; and detecting a presence or absence of said output, wherein said presence or absence of said output determines said molecular state of said cell.

11. The method of claim 10, wherein if said output is produced, the molecular state of the cell is a disease state.

12. The method of claim 10, wherein if said output is not produced, the molecular state of the cell is a disease state.

13. The cell or method of claim 11, wherein said disease state is cancer.

14. The plasmid of claim 1, wherein said output comprises one or more of a protein or an RNA molecule.

15. The plasmid of claim 14, wherein said RNA molecule comprises a miRNA (micro RNA) molecule.

16. The plasmid of claim 1, wherein said transcription factor binding region(s) comprise regions that are located in one or more of downstream, upstream or in-between conserved regions, or a combination thereof.

17. The plasmid of claim 1, wherein said transcription factor(s) comprise only innate transcription factor(s) already present in a cell.

18. The plasmid of claim 1, wherein said transcription factor(s) comprise prokaryotic transcription factors or eukaryotic transcription factors.

19. The plasmid of claim 1, wherein said transcription factors are repressing or activating transcription factors.

20. The plasmid of claim 1, wherein said prokaryotic transcription factor(s) comprise any of TetR, LacI, or λ-Repressor.

21. The plasmid of claim 1, wherein said eukaryotic transcription factor(s) comprise any of p53, E2F, or FOXO.

22. An autonomous synthetic programmable device adapted to determine a cell state according to at least three different predefined markers, comprising a transcription machinery and a regulatory element for regulating said transcription machinery, said regulatory element comprising at least three different binding regions, each binding region binding a different transcription factor and each binding region corresponding to a different predefined marker, wherein the regulatory element permits said transcription machinery to provide a transcription output if and only if all three different transcription factors are absent and wherein each transcription factor is capable of blocking transcription by said transcription machinery and hence blocking said transcription output.
